(19) United States
(12) Patent Application Publication
(10) Pub. No.: US 2011/0168188 A1
(43) Pub. Date: Jul. 14, 2011

(54) MANDIBULAR POSITIONING APPLIANCE

(76) Inventors: Richard W. Moore, Portland, OR (US); Joseph R. Zelk, Portland, OR (US)

(21) Appl. No.: 13/004,824

(22) Filed: Jan. 11, 2011

Related U.S. Application Data
(60) Provisional application No. 61/293,790, filed on Jan. 11, 2010.

(51) Int. Cl. A61F 5/56

(52) U.S. Cl. 128/848

(57) ABSTRACT

Embodiments herein provide a mandibular positioning appliance that is fully adjustable (adjustable in all planes). The appliance may be used for the management of sleep disordered breathing (SDB). The full spectrum of SDB, from the mildest snoring to severe obstructive sleep apnea (OSA), may be managed by the stabilization and features that this appliance offers. In embodiments, an appliance may be used by itself or may be used in conjunction with other therapies, such as positive airway pressure and/or a tongue retaining device.

### CROSS REFERENCE TO RELATED APPLICATIONS

The present application claims priority to U.S. Provisional Patent Application No. 61/293,790, filed Jan. 11, 2010, entitled “Mandibular Positioning Appliance,” the entire disclosure of which is hereby incorporated by reference in its entirety.

### TECHNICAL FIELD

Embodiments herein relate to the fields of sleep medicine and oral orthotics, and, more specifically, to a system for mandibular positioning and stabilization to facilitate proper and effective breathing during sleep.

### BACKGROUND

Various techniques have been used to treat sleep disordered breathing (SDB) patients. Typical positive airway pressure (PAP) therapy introduces pressurized air through the nose or through the nose and mouth to pneumatically “splint” the airway open to prevent narrowing or collapse of the airway during the tongue and oro-pharyngeal muscular relaxation or paralysis during sleep. Oral appliance therapy (OAT) treats sleep disordered breathing by repositioning and stabilizing the mandible using a removable dental orthotic during sleep. OAT reduces proximity of the tongue base and other tissues such as the pharyngeal walls, uvula, soft palate and epiglottis. This mechanical separation of tissues reduces airflow resistance and can eliminate the apneas (airway collapse) and hypopneas (airway narrowing) associated with SDB. In many cases, snoring can be effectively eliminated by such mechanical therapy.

Unfortunately, PAP therapy compliance is problematic associated with the inability to produce natural sleep, frequently due to the high pressures required to maintain airway patency throughout the night and the discomfort from wearing the PAP device. Fragmented sleep can result in residual daytime somnolence and poor compliance with therapy. The compliance of OAT compared to PAP is superior, but the variability of effectiveness is problematic.

Generally, oral appliances utilize the maxilla and maxillary teeth to anchor the mandible to a more forward open position to reposition the tongue and other pharyngeal structures forward. This results in a dilation of the pharynx in order to maintain patency of the airway during any neuro-muscular status that varies with the continuously changing stages of sleep.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates an exemplary cross-section of a mandibular positioning appliance in accordance with an embodiment;

FIGS. 2A and 2B illustrate perspective views of mandibular positioning appliances in accordance with embodiments;

FIGS. 3A and 3B illustrate perspective views of a mandibular positioning appliance that includes a tongue retaining device in accordance with an embodiment;

FIG. 4 illustrates an example of a PAP interface configured for coupling with a mandibular positioning appliance in accordance with an embodiment;

FIG. 5A illustrates a perspective view of an example of an oro-nasal PAP interface coupled with a mandibular positioning appliance in accordance with an embodiment;

FIG. 5B illustrates a perspective view of the embodiment of FIG. 5A showing the oro-nasal mask in dashed lines;

FIG. 5C illustrates an exploded view of the embodiment of FIG. 5A; and

FIG. 5D illustrates a cross-sectional view of a coupling mechanism in accordance with an embodiment.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact; however, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide a mandibular positioning appliance that is fully adjustable (adjustable in all planes). The appliance may be used for the management of sleep disordered breathing (SDB). The full spectrum of SDB, from the mildest snoring to severe obstructive sleep apnea (OSA), may be managed by the stabilization and features that this appliance offers. In embodiments, the appliance may be used by itself or may be used in conjunction with other therapies, such as positive airway pressure. Furthermore, in some embodiments, the appliance may include a tongue retaining device for retaining the tongue in a forward position to further treat SDB.

To optimize the dilation of the pharyngeal airway, the ability to vary both the amount of mandibular protrusion and the degree of mandibular opening (the facial vertical dimension) would be beneficial. The mandibular positioning appliance described herein provides such benefits, namely to easily and accurately adjust the relative position of the mandible. Further, the adjustment is manageable “chair-side” while the patient is under the direct care of a dental professional.

Embodiments herein may use suitable dental laboratory technology to prepare custom trays of thermo-formed single, dual laminate materials, or other suitable dental materials. In embodiments, these trays fit intimately to the teeth and provide both retention and stability for the appliance. In other embodiments, trays may be fit to fully or partially edentulous patients by utilizing one or more of the dentures, gums, lips, and/or cheeks to secure the appliance in addition to, or instead of, the teeth.

In embodiments, trays may be formed as independent maxillary and mandibular elements. Many different materials and techniques may be used to form such an appliance. Suitable materials for formation of the trays include, but are not limited to, one or more of denture acrylics, polycarbonate, polyurethane, polyester, copolymers thereof, and/or other similarly functioning materials.

In an embodiment, the upper and lower elements (maxillary and mandibular elements) of the mandibular positioning appliance may be coupled together by a heat-moldable material, such as a thermoformable polymer or thermoplastic, e.g., a low fusion temperature polymer. The heat-moldable material may provide a repeatably adjustable, stable material that securely and accurately couples the maxillary and mandibular elements and holds the mandible in a desired position, such as a desired therapeutic position. Polycaprolactone is one such material suitable for this purpose. When sufficient heat is applied to polycaprolactone (from about 140° F. to about 212° F.), the material softens, turns from opaque to clear, and permits adjustment of the spatial relationship of the maxillary and mandibular appliance elements. When the material cools, the defined spatial relationship is preserved. If, after cooling, the position is not as desired, the process may be repeated by reheating the polymer and readjusting the maxillary and mandibular spatial relationship. Reheating may be effected by any suitable means, such as immersion in a hot water bath, direct flame from a torch (blow torch, dental torch, micro torch), and/or another heat source.

The physical attributes of the coupling allow for easy and accurate adjustment of the relative mandibular position in all directions (all planes) and the entire three-dimensional space to attain optimal pharyngeal dilation. Embodiments herein provide adjustability of the amount of mandibular protrusion (longitudinal dimension), the horizontal mandibular position, and/or the degree of mandibular opening (vertical dimension). The amount of the heat-moldable material used to couple together the maxillary and mandibular elements may be varied as necessary to provide the desired mandibular opening.

Embodiments herein provide mandibular stabilization and positioning. A static position, albeit adjustable, provides a more stabilized pharyngeal musculature. Mandibular stability further reduces temporomandibular joint dysfunction, a common side effect of oral appliance therapy. In addition, in embodiments, an appliance as described herein reduces temporomandibular joint stress during side sleep as the alignment of the mandible is maintained against the force of gravity.

In various embodiments, the relative mandibular position may be adjusted incrementally over time to achieve optimal pharyngeal dilation while lessening discomfort to the patient. That is, during a first fitting, the mandibular element may be placed in a first position relative to the maxillary element. Once the patient has become accustomed to the first position, the mandibular element may be moved to a second position relative to the maxillary element during a second fitting. The mandibular element may be further adjusted in a similar manner during subsequent fittings until optimal pharyngeal dilation is achieved. The position adjustments may be separated by any suitable period of time, such as days, weeks, or months. The heat-moldable material allows for repeated adjustments by repeating the heating, positioning, and cooling, as explained above. This process of incremental adjustment may lessen the discomfort that may otherwise result from realignment of the mandibular position.

In some embodiments, the heat-moldable material may be disposed between the maxillary and mandibular elements only in posterior portions of the maxillary and mandibular elements. The use of a polymeric coupling only between the posterior portions of the maxillary and mandibular elements, rather than mechanical devices that can impede tongue space, permits more complete tongue repositioning by providing increased available space in the anterior portion. In addition, maintaining spatial adjustability of the mandibular element without mechanical devices such as screws, rods, elastic straps or other such devices, may reduce risks of swallowing or aspirating parts dislodged during clenching, grinding, and/or other nocturnal parafunctional events. With the ability to provide a wide open oral airway, the tongue reflexively moves forward into that opening and passive retention of the tongue in the opening may occur. This advancement of the dorsum of the tongue further enhances the airway dilation. Such embodiments also provide enhanced speech quality, the ability to drink, and the ability to moisten the lips with the tongue, all with the appliance in place. In addition, such an embodiment reduces claustrophobia since the tongue has near full function/movement.

In other embodiments, the heat-moldable material may be disposed between all or substantially all of the surfaces between the maxillary and mandibular elements. In such embodiments, air holes may be provided to allow breathing through the mouth and/or to allow the use of the mandibular positioning appliance with a PAP system. However, in some embodiments, no air holes may be included in the heat-moldable material to encourage the patient to breathe through the nose. In yet other embodiments, any arrangement of the heat-moldable material between the maxillary and mandibular elements may be utilized.

In an embodiment, the mandibular positioning device may further include a tongue retaining device to retain the tongue in a forward position, augmenting the attempt to prevent the tongue from blocking the airway of the patient. In some patients, during deep sleep, the tone of the tongue muscle and voluntary airway muscles can be lost, causing partial or complete interference with the airway. The tongue retaining device may be especially beneficial for these patients. In an embodiment, the tongue retaining device may include a bulb that defines a cavity between the anterior portion of the maxillary and mandibular elements and extending outward. The bulb may be configured so that in use, the patient may place the tongue in the cavity and create a reduced pressure in the cavity by sucking out the air as the tongue is introduced into the cavity. The reduced pressure in the cavity may produce a vacuum effect that helps retain the tongue in the cavity. Additionally, or alternatively, the bulb may include mechanical elements which retain the tongue in the forward position, such as ridges, slopes and/or other mechanical elements. For example, the bulb may be narrower at a posterior portion, closer to the base of the tongue, than at an anterior portion further from the base of the tongue. The narrower posterior portion may facilitate creating a seal with the tongue in addition to mechanically retaining the tongue in the forward position.

In an embodiment, the tongue retaining device may be made from a heat-moldable material. By using a heat-moldable material, the tongue retaining device may be formed to fit the patient's mouth and/or tongue. The personalized fit may provide an improved pneumatic seal with the tongue, as well as enhanced comfort. As the heat-moldable material cools, the bulb may be slightly compressed to provide additional mechanical retention of the tongue. The heat-moldable material used to produce the tongue retaining device may be the same material used to couple together the maxillary and mandibular elements, or may be a different heat-moldable material. In some embodiments, the mandibular positioning device with tongue retaining device may not include air holes in order to allow the negative pressure in the cavity to hold the tongue in place. The device may also encourage the patient to breathe through the nose, providing a number of advantages. For example, humans are designed for nasal breathing, since the air is warmed and humidified in preparation for the lungs. Additionally, there is a reflexive tongue advancement associated with normal nasal breathing which clears the airway as described above. Furthermore, the efferent nerve sensors that provide information to the medulla of the brain to control breathing are found primarily in the nasal mucosa. Therefore, in patients with a competent nasal airway, the addition of the sealed tongue retaining device may be advantageous. Should the nose become congested or impaired, the patient may open the mouth to allow air to enter through the mouth.

Alternatively or in addition, in some embodiments, the mandibular positioning device with tongue retaining device may include one or more air holes to allow breathing and/or to configure the mandibular positioning device for use with a PAP interface. In some such embodiments, the air holes may be located in a separate portion from the bulb in order to maintain the seal of the bulb.

In an embodiment there is provided a hybridized positive airway pressure system (HPAP) combining PAP for efficacy of airway maintenance with mandibular advancement/stabilization using a mandibular positioning appliance. In such an embodiment, the mandibular positioning device as described herein may be further configured to couple with a PAP interface. Similarly, the PAP interface may be configured to couple with the mandibular positioning device. The combination of mandibular positioning using the mandibular positioning appliance with PAP may eliminate/reduce straps, reduce leaks, reduce the required applied pressure, improve sleep quality, reduce claustrophobia, eliminate/reduce headaches, and/or prevent aerophagia, drying of eyes, chronic sinusitis, and/or pressure ulceration of the skin, etc. Dilation of the airway mechanically using such an appliance reduces the level of air pressure required to maintain airway patency. Furthermore, embodiments may provide a mandibular positioning device configured to couple with a PAP interface without the need for straps around the patient's head. The stability of the mask coupled to the mandibular positioning device facilitates side sleep posture which promotes reduced PAP pressures since the pressure of the pillow is less likely to disrupt the interface seal against the skin. Nocturnal parafunctional mandibular activity is found in a high percentage of sleep apnea patients. The use of this progressive system of therapy is an advantage over the use of PAP alone because the significant loss of tooth structure from the clenching and grinding of the teeth at night can be eliminated. Further, the spreading of the anterior teeth with resultant functional and aesthetic consequences, caused by the air pressure behind the tongue using PAP therapy, may be prevented by using the oral appliance first as advocated in this progressive system of therapy.

In embodiments, the PAP interface may be any suitable airway interface. For example, the PAP interface may include a nasal interface that is configured to fit within, on, and/or around the nares of the nose. Alternatively, the PAP interface may include an oro-nasal interface, such as a mask, that covers the mouth and the nares of the nose. Accordingly, in embodiments, a suitable airway interface may be a mask configured primarily for external application to the nose/face, may be or may include one or more tubes, “pillows,” or other similar devices for fitting within the nares, or may be a combination of various approaches.

In an embodiment, the mandibular positioning appliance may be fixed to or removably coupled to the PAP interface. Accordingly, the mandibular positioning appliance may be configured to couple with the PAP interface. For example, the mandibular positioning appliance may include a receiving element configured to receive an adjustment mechanism of the PAP interface. In some embodiments, the adjustment mechanism may include a male threaded screw element, and the receiving element may include a female threaded element, although other coupling mechanisms are also possible. In some embodiments, the receiving element may be coupled to the mandibular positioning appliance by a heat-moldable material. By using the heat-moldable material, the location of the receiving element may be adjusted to improve the seal of the PAP interface when in use and/or to improve the comfort of the patient. When the PAP interface is coupled rigidly to the skeleton via the teeth, as it is in embodiments herein, rather than non-rigidly coupled to the skin of the face via straps, the most common cause of leakage, i.e., from movement of the body, may be eliminated.

In some embodiments, the adjustment mechanism may be coupled to a housing coupled to the PAP interface. The adjustment mechanism may or may not be removable from the housing. The adjustment mechanism may form a seal, such as a pneumatic seal, with the mask. In some embodiments, the receiving element may be pivotable in the housing to allow the mask of the PAP interface to self-equalize as the mask contacts the face of the user. That is, the mask may be adjustable in a plurality of planes with respect to the mandibular positioning appliance. As the mask contacts the face of the user, the orientation of the mask may adjust to distribute the pressure on the face evenly as a direct result of the pivotability of the receiving element. This self-equalization enhances the comfort to the user and the seal of the mask on the user's face. In some embodiments, the receiving element may be designed to provide some motion with respect to the adjustment mechanism to allow adjustment of the mask position relative to the mandibular positioning appliance. Furthermore, in some embodiments, the adjustment mechanism may be pivotable within the housing while still maintaining a seal with the housing.

When the adjustment is complete, the pressure of the mask against the skin provides enhanced stability of the mask when in use. The pressure may reduce and/or eliminate the pivoting of the receiving element once the mask is in place. The design of the housing and the coupling mechanism provide for disassembly of the components of the mask to facilitate cleaning and maintenance. In some embodiments, if the coupling mechanism is rotated beyond the detent position, the coupling mechanism may be uncoupled from the housing and/or mask.

In some embodiments, the receiving element may be disposed on a support arm extending upward and/or outward from the mandibular positioning appliance. The support arm may be flexible to enhance comfort and allow settling of the mask on the face, while still being rigid enough to support the receiving element when coupled to the mask. For example, the support arm may be made from a flexible material, such as polycarbonate, acrylic/polyester copolymers, nylon, resilient metal or a wide variety of other materials. Additionally, or alternatively, the support arm may be constructed to provide some flexibility in one or more directions. For instance, the support arm may have a length that is relatively long compared to a thickness, which may provide some flexibility. Furthermore, in an embodiment, a vertical design of the support arm may advantageously baffle the direct flow of air from the PAP interface to the mouth. This design reduces drying of the mouth that may otherwise occur.

Additionally, the support arm allows the receiving element and adjustment mechanism to be disposed in any suitable region of the mask. That is, a central axis of the receiving element and adjustment mechanism may not lie in a central plane of the mandibular positioning appliance, i.e., a plane running substantially between the maxillary and mandibular elements. This may enhance the stability of the mask.

Some embodiments may include a method of progressive treatment wherein the patient may begin treatment with a mandibular positioning appliance, and progress to additional methods of treatment as needed. In an embodiment, the mandible may be stabilized using a mandibular positioning appliance as described herein. In some embodiments, the advancement of the mandible by the mandibular positioning appliance may be done incrementally over a plurality of adjustments. After the patient is provided with the mandibular positioning appliance, the progress of the patient's airway flow and sleep quality may be monitored. If the appliance is insufficiently managing the patient's sleep disordered breathing, at least one of a tongue retaining device, a nasal PAP interface, and/or an oro-nasal PAP interface may be added. Each subsequent treatment may be added using the same mandibular positioning appliance that was already fitted to the patient. Alternative or additional treatments may be added later as needed. Similarly, one or more of the treatment devices may be removed from the mandibular positioning appliance as desired.

For example, if the patient's treatment has not progressed sufficiently using only the mandibular positioning appliance, a tongue retaining device may be added to the mandibular positioning appliance. In some cases, that may sufficiently improve the patient's sleep disordered breathing. However, if the patient is still not adequately treated, a PAP interface may be provided that is configured to be coupled with the mandibular positioning appliance. At this point, a receiving element may be added to the mandibular positioning appliance for receiving the PAP interface.

The tongue retaining device may or may not be removed prior to adding the PAP interface. For some patients, such as those with trouble breathing through their noses during sleep, the step of providing a tongue retaining device may be skipped altogether. For these patients, a PAP interface may be added if the mandibular positioning appliance alone is not sufficient.

The PAP interface may be any suitable interface, such as a nasal PAP interface or an oro-nasal interface. In some cases, the patient may first be provided with a nasal PAP interface. If the nasal PAP interface does not sufficiently improve the patient's sleep disordered breathing, an oro-nasal interface may be provided. The oro-nasal interface may be indicated for patients for whom the nasal mask alone does not provide effective treatment. The reasons for ineffective airway management with the nasal mask alone may include, but are not limited to, an impaired and incompetent nasal airway due to history of trauma, chronic congestion, sinusitis, and/or aberrant nasal anatomy. The oro-nasal mask may also be required to solve the leak of pressure from the mouth that typically accompanies these nasal airway deficiencies, since the mouth may open in sleep to compensate for the decreased nasal competency. If the mouth opens during sleep, the nasal air pressure alone may be ineffective sleep apnea therapy.

Throughout the progressive treatment, the mandibular positioning appliance retains the ability to make further mandibular positioning adjustments as needed because of the heat-moldable material that couples together the maxillary and mandibular elements. Furthermore, the heat-moldable material allows each subsequent treatment device to be added or subtracted if and when it is needed while utilizing the same mandibular positioning appliance.

FIG. 1 illustrates an exemplary cross-section of a mandibular positioning appliance in accordance with an embodiment. A maxillary element 102 is coupled to a mandibular element 104 by an intermediate material 106. Material 106 may be a heat-moldable material or low fusion temperature polymer, such as polycaprolactone.

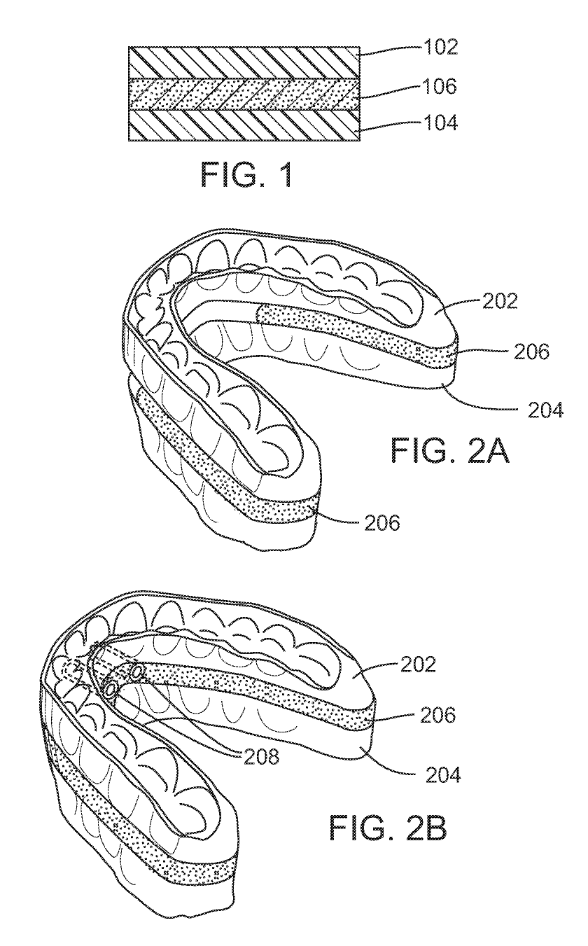

FIGS. 2A and 2B illustrate perspective views of mandibular positioning appliances in accordance with embodiments. Maxillary element (tray) 202 is coupled to mandibular element (tray) 204 by an intermediate material 206. Material 206 may be a heat-moldable material or low fusion temperature polymer, such as polycaprolactone. FIG. 2A illustrates an example in which material 206 is only positioned between the posterior portions of maxillary element 202 and mandibular element 204. In FIG. 2A, a relatively large open space is preserved in the anterior portions between maxillary element 202 and mandibular element 204. This allows advancement of the dorsum of the tongue, which further enhances the airway dilation. Such embodiments also provide enhanced speech quality, the ability to drink, and the ability to moisten the lips with the tongue, all with the appliance in place. Alternatively, FIG. 2B illustrates an example in which material 206 is positioned between all or substantially all of the surfaces between maxillary element 202 and mandibular element 204. With this configuration, through holes 208 may be provided. Through holes 208 may enhance the management of saliva and/or provide air passage. Other configurations of air holes or other such passageways may be provided as desired. In addition, while FIGS. 2A and 2B illustrate two alternatives for configuration of a material between separate maxillary and mandibular elements, other configurations may be provided in light of the teachings herein. In an embodiment, because the appliance is constructed from separate maxillary and mandibular elements, replacement of single maxillary or mandibular elements is permitted without the replacement of the entire device.

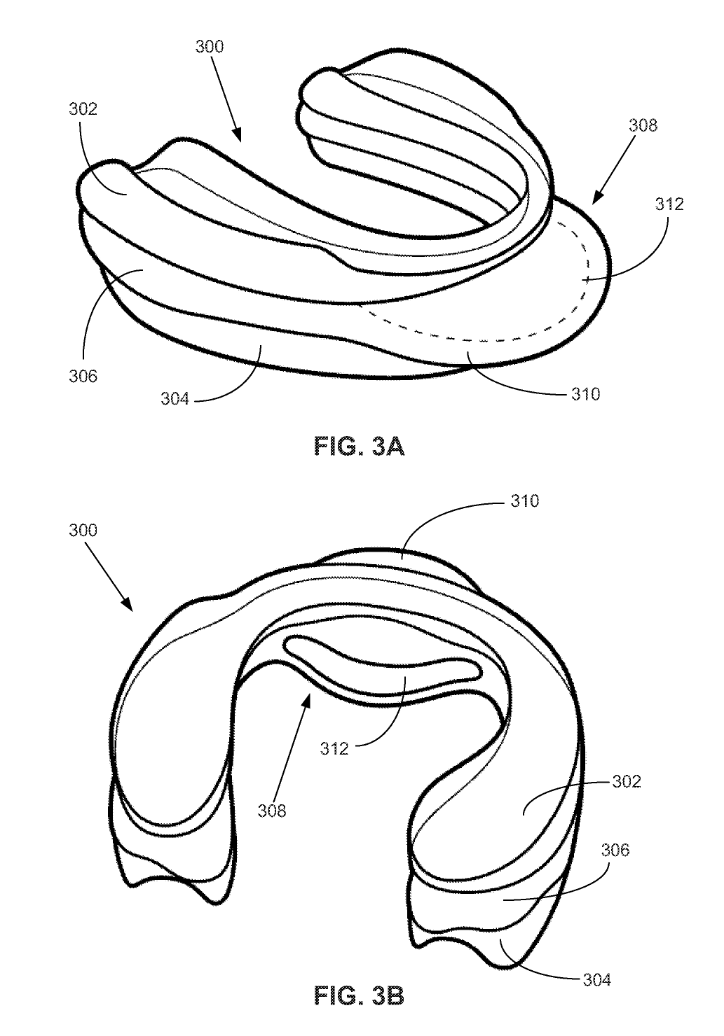

FIGS. 3A-B illustrates an example of a mandibular positioning device 300 that includes a tongue retaining device 308 in accordance with an embodiment. Mandibular positioning device 300 includes a maxillary element 302 and a mandibular element 304, which are coupled together by an intermediate material 306. Intermediate material 306 may be a heat-moldable material, such as polycaprolactone. Tongue retaining device 308 is also formed by intermediate material 306. Tongue retaining device includes a bulb 310 that defines a cavity 312. Bulb 310 is configured so that the patient may create a reduced air pressure in cavity 312 by sucking in air as the tongue is introduced into cavity 312. Cavity 312 may be molded to the patient's tongue so that the tongue may create a pneumatic seal with cavity 312. Bulb 310 may also be compressed slightly during molding to create a mechanical retention mechanism which may aid in retaining the patient's tongue in cavity 312.

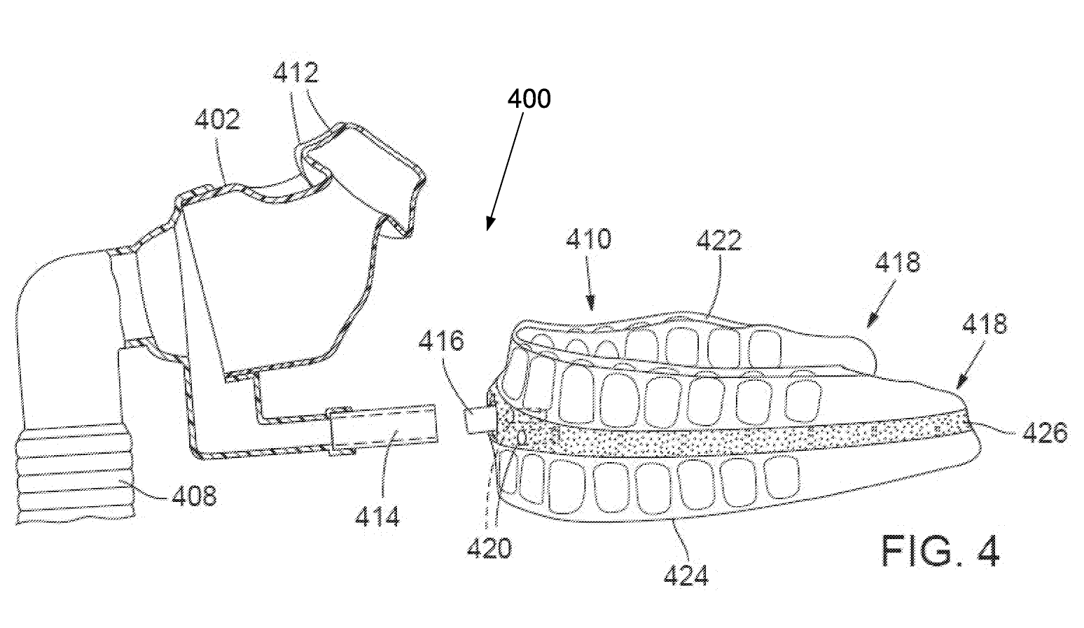

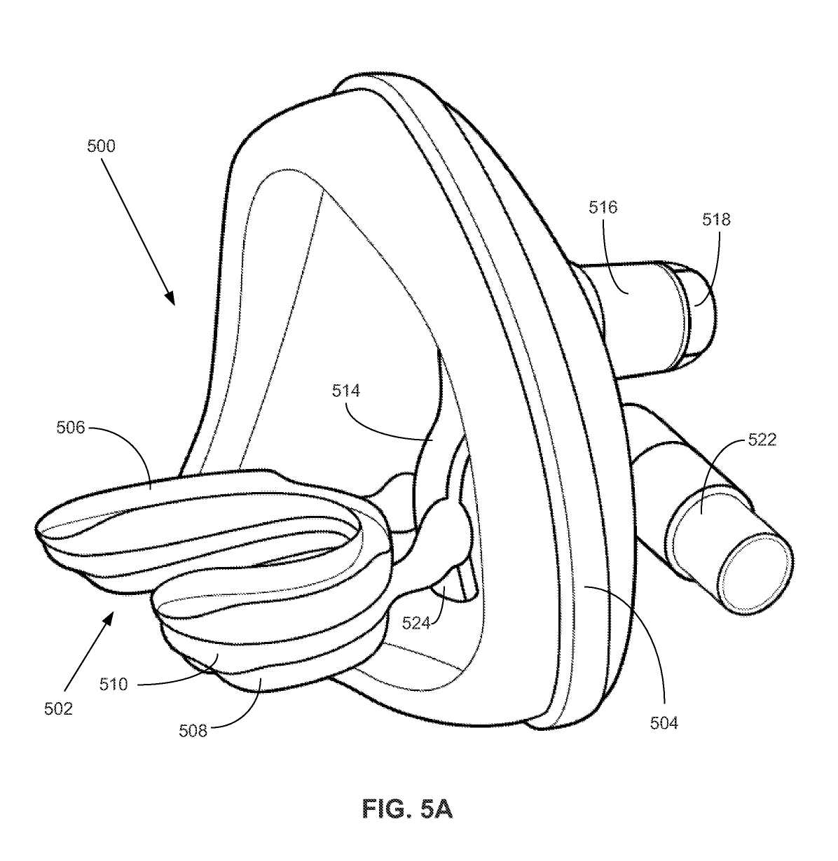

FIG. 4 illustrates an example of a PAP interface configured for coupling with a mandibular positioning appliance in accordance with an embodiment. Device 400 is shown with airway interface 402 configured for placement within the nares of an individual. Airway interface 402 is coupled at one end to a hose 408 that may in-turn be coupled to a source of pressurized air (such as a pump). Airway interface 402 is also coupled to a mandibular positioning appliance 410 configured to engage with the teeth of the individual. Airway interface 402 may have any of a variety of mechanisms for delivering air to the individual, including full coverage nasal masks and/or pads/pillows 412. As used herein, the term “pillows” refers to intranasal seals that inflate and seal at the rims of the nostrils.

Airway interface 402 may be coupled to mandibular positioning appliance 410 using any suitable mechanism. In FIG. 4, airway interface 402 has an extended mechanism 414 configured to couple with a post 416 on appliance 410. Other mechanisms may be utilized in embodiments.

Appliance 410 may be configured to engage with the teeth of the individual. Appliance 410 has a maxillary element 422 coupled to a mandibular element 424 by a material 426. Material 426 may be a thermoformable polymer to permit adjustment of the relative positioning of the maxillary and mandibular elements when the polymer is in a heated and moldable state.

In an embodiment, appliance 410 may have at least one posterior flange 418 configured to assist with sealing the oral cavity when appliance 410 is in place. In an embodiment, flange(s) 418 may be constructed from the same material as maxillary element 422 and mandibular element 424 or the same as material 426, or may be another material.

In an embodiment, oral appliance 410 may have one or more holes 420 that provide a passage for saliva between the oral and labial cavities for swallowing, drooling, and/or to provide for a certain amount of air passage.

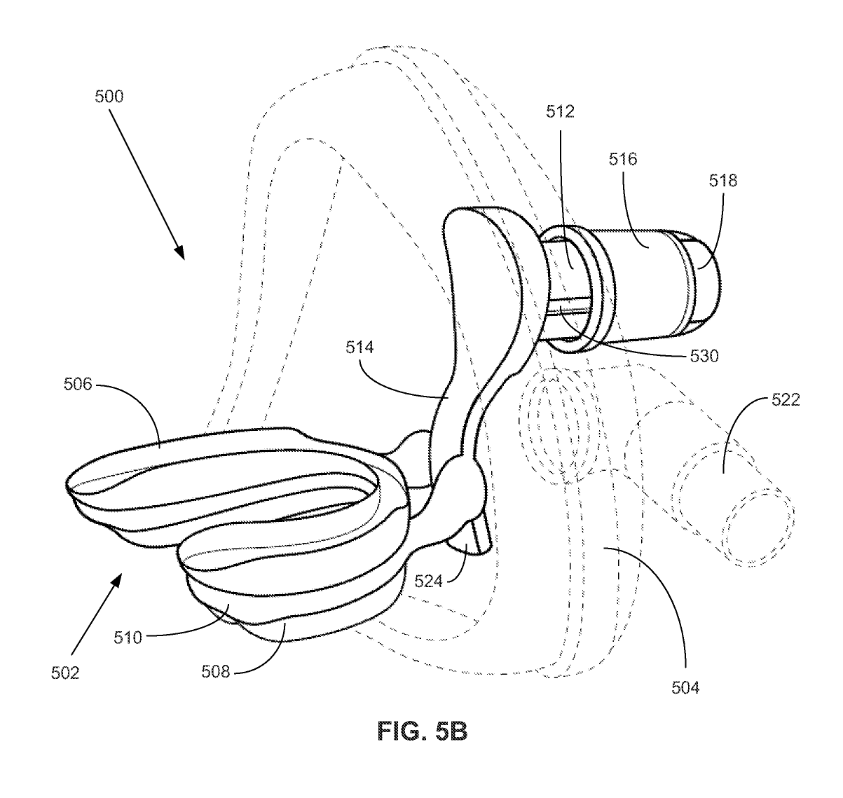

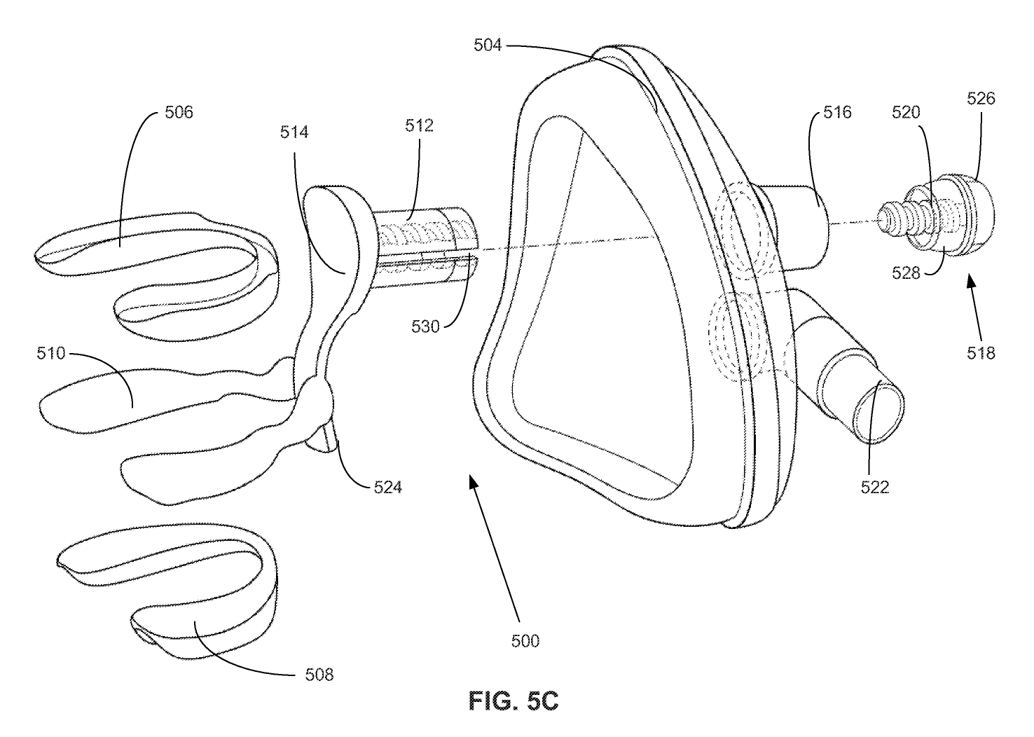

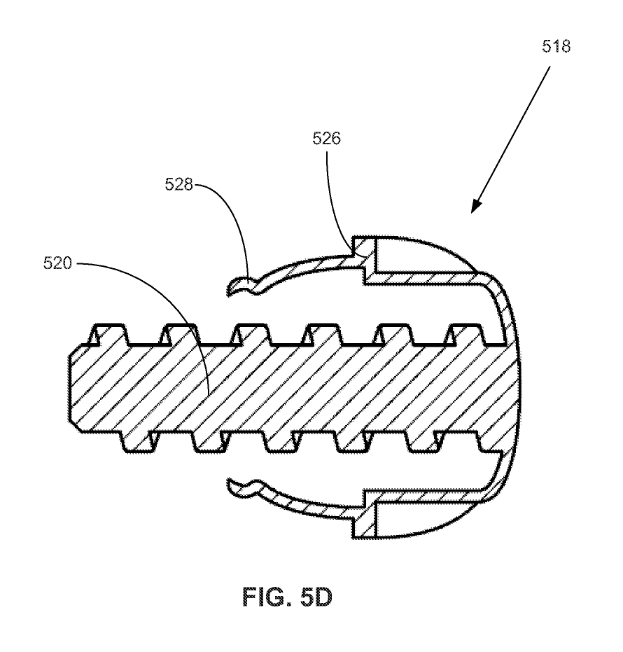

FIGS. 5A-C show an embodiment of a hybrid PAP device 500 including a mandibular positioning device 502 and a mask 504. Mandibular positioning device 502 includes a maxillary element 506, a mandibular element 508, and a heat-moldable material 510 coupling maxillary element 506 to mandibular element 508. Mandibular positioning device 502 further includes a receiving element 512 disposed on a support arm 514. Support arm 514 is coupled to the heat-moldable material 510. Mask 504 includes a housing 516 configured to receive an adjustment mechanism 518. Adjustment mechanism 518 includes a screw element 520 configured to couple with receiving element 512. Mask 504 further includes a gas tube interface 522 for coupling the mask to a gas source, such as air. Support arm 514 includes a baffle portion 524 that may prevent direct flow of gas from the gas tube interface 522 to the mouth of the user. This may decrease drying of the mouth. Furthermore, a central axis of the receiving element 512 is disposed above a central plane of the mandibular positioning appliance 502 (i.e., a plane running substantially between maxillary element 506 and mandibular element 508). This may enhance the stability of the mask.

FIG. 5D shows a cross-sectional view of adjustment mechanism 518 including the screw element 520. Adjustment mechanism 518 includes a flange 526 configured to create a seal, such as a pneumatic seal, with the housing 516 shown in FIG. 5C. Adjustment mechanism 518 further includes a flexible extension 528 that provides a functional limit to the extension of the mask away from the face.

Referring now to FIGS. 5A-C, hybrid device 500 allows for changing the position of the mask 504 relative to the face of the user. In the embodiment shown in FIGS. 5A-C, the adjustment mechanism utilizes a male threaded screw and the receiving element is a female threaded piece, although other configurations are possible. With the mandibular positioning appliance 502 placed on the teeth of the user, as the adjustment mechanism 518 is turned clockwise from an observer's perspective, the mask is moved toward the face of the user and the pressure of the mask 504 against the skin is increased until the fit of the mask against the skin is optimized for comfort and/or leak reduction/elimination. When the adjustment mechanism 518 is turned counter-clockwise from an observer's perspective, the mask 504 moves away from the face of the user. If significant additional counterclockwise force is applied, the adjustment mechanism 518 may be uncoupled from the receiving element 512. The adjustment mechanism 518 may also be removed from the mask 504.

Receiving element 512 is pivotable in housing 516 when engaging the adjustment mechanism (male element) 518 and during operation of the adjustment mechanism throughout its range of operation. This pivoting feature allows for self-equalization of the seal of mask 504 for comfort and effectiveness. The receiving element 512 may be designed to allow some motion with respect to the screw element 520 to allow the receiving element 512 to pivot. Receiving element 512 may include a gap 530 to provide added flexibility. Furthermore, support arm 514 may be flexible to allow self-equalization of the fit of the mask 504. Additionally, since the receiving element is coupled to the mandibular positioning device by the heat-moldable material 510, the position of the receiving element may be adjusted as needed. This may be done when the patient is first fitted for the mask, or at a later time.

Furthermore, the use of heat-moldable material 510 facilitates the progressive approach to treatment by allowing the coupling mechanism to be added to the mandibular positioning device after the patient has first used the mandibular positioning device on its own. The receiving element 512 and support arm 514 may be added to a stand-alone mandibular positioning device by adding and/or re-heating the heat-formable material 510 and attaching the support arm 514.

The design of the hybrid PAP device 500 is advantageous by utilizing the benefits of the mandibular advancement device as listed earlier in this application. In addition, for those patients for whom the mandibular advancement device alone is insufficient treatment, the ability to augment those effects with a simplified, more stable and comfortable mask enhances patients' likelihood of normalized airway function in sleep and resolution of the deleterious effects of obstructive apnea. Further, the likelihood of managing central and mixed sleep apneas is also significantly enhanced.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A device for managing sleep disordered breathing, comprising:
a mandibular positioning appliance configured to engage with the mouth and/or teeth of an individual, the mandibular positioning appliance comprising a maxillary element coupled to a mandibular element by a heat-moldable material in sufficient quantity to permit repositioning of the mandibular element with respect to the maxillary element when the heat-moldable material is in a heated state and to secure the maxillary element to the mandibular element when the heat-moldable material is in a non-heated state.

2. The device of claim 1, wherein the heat-moldable material is positioned only between posterior portions of the maxillary element and the mandibular element leaving an anterior opening in the appliance between the maxillary element and the mandibular element.

3. The device of claim 1, wherein the heat-moldable material forms a tongue retaining device coupled to the maxillary element and the mandibular element, the tongue retaining device including a bulb that defines a cavity, wherein the cavity is configured to receive a tongue of the individual.

4. The device of claim 3, wherein the bulb is narrower at a posterior portion of the bulb than at an anterior portion of the bulb.

5. The device of claim 1, wherein the mandibular positioning appliance is configured to be coupled with at least one of a nasal airway interface and an oro-nasal airway interface.

6. A device for managing sleep disordered breathing comprising:
a mandibular positioning appliance configured to engage with a mouth and/or teeth of an individual;
an airway interface including an oro-nasal mask configured to be coupled to a source of input gas;
an adjustment mechanism configured to adjustably couple the oro-nasal mask to the mandibular positioning appliance; and
a receiving element coupled to the mandibular positioning appliance and configured to receive the adjustment mechanism;
wherein the oro-nasal mask is adjustable in a plurality of planes relative to the mandibular positioning appliance.

7. The device of claim 6 wherein the receiving element is pivotable relative to the oro-nasal mask when the receiving element is coupled to the adjustment mechanism.

8. The device of claim 6 wherein a longitudinal axis of the receiving element does not lie in a central plane of the mandibular positioning appliance.

9. The device of claim 6 wherein the mandibular positioning appliance comprises:
a maxillary element configured to engage maxillary teeth of the individual;
a mandibular element configured to engage mandibular teeth of the individual;
a heat-moldable material coupling the maxillary element to the mandibular element.

10. The device of claim 6 wherein the receiving element is coupled to a support arm and the support arm is coupled to the heat-moldable material.

11. The device of claim 10 wherein the support arm is configured to impede a direct flow of gas from the input gas supply to the mouth of the individual.

12. The device of claim 10 wherein the support arm is flexible.

13. A device for managing sleep disordered breathing, comprising:
an airway interface configured to be coupled to a source of input gas; and a mandibular positioning appliance coupled to the airway interface and configured to engage with a mouth and/or teeth of an individual, the mandibular positioning appliance comprising a maxillary element coupled to a mandibular element by a heat-moldable material.

14. The device of claim 13 wherein the airway interface includes at least one of a nasal strapless mask and an oro-nasal strapless mask.

15. The device of claim 13 wherein the mandibular positioning appliance further includes a tongue retaining device formed from the heat-moldable material.

16. A method for managing sleep disordered breathing in a patient, comprising:
fitting the patient with a mandibular positioning appliance, the mandibular positioning appliance configured to engage with a mouth and/or teeth of an individual and comprising a maxillary element coupled to a mandibular element by a heat-moldable material, wherein the fitting comprises:
heating the heat-moldable material to a heated state;
positioning the mandibular element in a first position relative to the maxillary element; and
cooling the heat-moldable material to a non-heated state.

17. The method of claim 16 further comprising determining the patient's sleep improvement as a result of the patient utilizing the mandibular positioning appliance for a period of time, and modifying a treatment based on the determining.

18. The method of claim 17 wherein the modifying comprises providing at least one of: a nasal PAP system to couple with the mandibular positioning appliance, an oro-nasal PAP system to couple with the mandibular positioning appliance, and a tongue retaining device to couple with the mandibular positioning appliance, to provide a combination therapy.

19. The method of claim 16, further comprising:
re-heating the heat-moldable material to a heated state;
positioning the mandibular element in a second position relative to the maxillary element, the second position being different than the first position; and
cooling the heat-moldable material to a non-heated state.
